(19) United States
(12) Patent Application Publication
(10) Pub. No.: US 2024/0207881 A1
(43) Pub. Date: Jun. 27, 2024

(54) AEROSOL GENERATOR CORE

(71) Applicant: STAMFORD DEVICES LIMITED, GALWAY (IE)

(72) Inventor: Michael TIMOTHY, Galway (IE)

(73) Assignee: STAMFORD DEVICES LIMITED, GALWAY (IE)

(21) Appl. No.: 18/551,731

(22) PCT Filed: Mar. 16, 2022
(86) PCT No.: PCT/EP2022/056887

§ 371 Date: Sep. 21, 2023

(30) Foreign Application Priority Data
Mar. 22, 2021 (EP) 21164099.0

(51) Int. Cl. B05B 17/06; A61M 11/00; B06B 1/06

(52) U.S. Cl. CPC B05B 17/0607; B06B 1/0688; A61M 11/005; B06B 2201/77

(57) ABSTRACT

An acrosol generator core (1) has an integral body (2) and membrane (4), and a vibratory drive connected to an electrical voltage supply. In some examples the body provides the vibration drive and comprises a piezoelectric polymer (2) of P(VDF-TrFE) material. In this manner a single manufacturing operation can provide the membrane. the support. and the vibration drive. and operations such as attaching a membrane to an annular support are avoided.

### INTRODUCTION

### Field of the Invention

The present invention relates to vibrating membrane aerosol generators for nebulizers.

### Prior Art Discussion

Aerosol generators which operate on the perforated membrane principle typically have an aperture plate with through holes, which can have a diameter in the region of several microns.

In many cases the vibrating membrane is provided by an aperture plate supported around its rim by a vibrating support which is vibrated by a piezo element. The membrane is used for aerosol delivery of liquid formulations delivering a controlled liquid droplet size suitable for pulmonary drug delivery. It is desirable to achieve a consistent and accurate particle size in combination with an output rate that can be varied to deliver the drug to the targeted area as efficiently as possible. Delivery of the aerosol to the deep lung such as the bronchi and bronchiole regions requires a small and repeatable particle size typically in the range of 2-4 μm. In general, output rates of greater than 0.1 ml/min are preferred.

Currently, aperture plates are produced by a variety of different means, including electroplating and laser drilling.

U.S. Pat. No. 6,235,177 (Aerogen) describes an approach based on electroplating, in which a wafer material is built onto a mandrel by a process of electro-deposition where the liquefied metals in the plating bath (typically Palladium and Nickel) are transferred from the liquid form to the solid form on the wafer. After the conclusion of the plating process, the mandrel/wafer assembly is removed from the bath and the wafer peeled from the mandrel for subsequent processing into an aperture plate.

WO2011/139233 (Agency for Science, Technology and Research) describes a micro-sieve manufactured using SU8 material with photo-masking. U.S. Pat. No. 4,844,778 (Stork Veco) describes manufacture of a membrane for separating media, and a separation device incorporating such a membrane. EP1199382 (Citizen watch Co. Ltd.) describes a production method for a hole structure in which there is exposure to photosensitive material in multiple cycles to provide deeper holes tapered towards the top because there is exposure through the first holes.

The aperture plate, however manufactured, is secured to a support such as a washer-shaped support, the attachment being for example brazing or adhesive. This is a potential point of failure due to the high-frequency drive applied to the aperture plate.

US2015/0136874 (Yu et al) describes a mesh structure and a vibrator structure are manufactured as one object, using etching or deposition, thereby avoiding need to couple a vibrator and a mesh.

US2011/0284656 (Murata) describes an atomizing member in which a vibrating membrane, a cylindrical piezoelectric ceramic body and a flange are integrally formed.

U.S. Pat. No. 5,518,179 (The Technology Partnership) describes a membrane which is integrally formed with the substrate of an electroacoustic actuator, the actuator being an electrostrictive piezoelectric or a magnetostrictive member.

The invention is directed towards providing improved manufacturing of an aerosol generator, in terms of being simpler and/or providing a nebulizer which is more reliable.

### SUMMARY

We describe an aerosol generator core comprising a body having a support supporting an integral membrane, and a vibration drive adapted to be connected to an electrical voltage supply.

Preferably, the vibration drive comprises the body, the body being configured to vibrate with application of an electrical voltage across it. Preferably, the body comprises a piezoelectric polymer. Preferably, the piezoelectric polymer comprises PVDF. Preferably, the body comprises a PVDF copolymer. Preferably, the piezoelectric polymer comprises (P(VDF-TrFE). Preferably, the piezoelectric polymer comprises P(VDF-TFE). In some examples, the body comprises a composite material comprising a polymer (e.g. PI, PC, PEEK) loaded with a secondary material (e.g. PZT or similar) that imparts piezoelectric properties on the body. Preferably, the composite material comprises a polymer selected from one or more of PI, PC, and PEEK and the secondary material comprises PZT.

In various examples the core comprises electrodes on a surface of the vibration drive. Preferably, at least one electrode comprises a metallisation layer. Preferably, at least one electrode has a characteristic of being applied using sputtering of one or more of copper, nickel or aluminium. Preferably, at least one electrode has a characteristic of being screen-printed. In some examples, at least one electrode has a characteristic of being screen printed with silver.

Preferably, at least one electrode has a thickness in the range of 10 nm to 20 μm. Preferably, the core has an outer lateral dimension between 5 mm and 50 mm, and optionally the core has a substantially disc shape and the lateral dimension is the diameter. Preferably, the lateral dimension is between 10 mm and 25 mm. Preferably, the membrane diameter is between 1 mm and 50 mm, more preferably between 2 mm and 10 mm. Preferably, the core body thickness is between 20 μm and 5000 μm, more preferably between 30 μm and 1500 μm. Preferably, the membrane thickness is between 10 μm and 300 μm, and more preferably between 30 μm and 150 μm.

In some examples the core further comprising a discrete vibration drive element providing a vibration drive which is additional to the vibration drive provided by the body. The discrete vibration element may be embedded within the body. The discrete vibration element may be supported on a surface of the body.

The vibration drive may comprise solely a discrete drive element, in one example a piezoelectric drive element. The discrete drive element may be seated in a groove in the support.

We also describe an aerosol generator comprising a core of any example described herein, and power conductor terminals for delivering electrical power to the vibration drive. The power conductor terminals may comprise at least one spring pin.

In some examples the power conductor terminals comprise a spring pin for conducting power to a first vibration drive surface and another spring pin for conducting power to an opposed vibration drive surface via a clip-shaped terminal which extends around the body at a side edge. In a preferred example the clip-shaped terminal comprises a first limb engaged by a spring pin and pressing against an insulator, said first limb being connected to a second limb which provides power to the opposed vibration drive surface.

The power conductor terminals may comprise a spring pin for conducting power to a first vibration drive surface and another spring pin for conducting power to an opposed vibration drive surface via a plated through hole. The spring pins may be parallel to each other and both on an upstream side or a downstream side of the core.

We also describe a method of manufacturing an aerosol generator core of any example described herein, the method comprising forming the body by any one or more of hot embossing, micro-moulding, inkjet printing, 3D Printing, or UV Nanoimprint Lithography.

In some examples, the method includes hot embossing in which temperature of a polymer sheet is raised above its melting range followed by pressing a heated mould into the polymer to fill surface structures.

In some examples, the method includes injection moulding in which polymer granules are melted, and then injected into a mould under pressure where it solidifies.

In some examples, the method includes Nanoimprint Lithography (NIL) to provide features on a polymer surface with a pattern via deformation onto a resist material using a mould. In some examples, the method includes transfer moulding using thermosetting epoxy resin.

We also describe an aerosol generator core comprising an integral support body having an annular shape and a central integral membrane of smaller thickness than the body, and a vibration drive adapted to be connected to an electrical voltage supply.

In one example, the vibration drive is integrated in the support body. In one example, the body comprises a piezoelectric polymer. In one example, the piezoelectric polymer comprises PVDF.

In one example, the piezoelectric polymer comprises (P(VDF-TrFE). In one example, the piezoelectric polymer comprises P(VDF-TFE).

In one example, the core has a diameter between 5 mm and 50 mm, preferably between 10 mm and 25 mm, for example 15 mm. In one example, the membrane diameter is between 1 mm and 50 mm, preferably between 2 mm and 10 mm diameter, for example 4 mm. In one example, the core body thickness is between 20 μm and 5000 μm, preferably between 30 μm and 1500 μm, for example 500 μm. In one example, the membrane thickness is between 10 μm and 300 μm, preferably between 30 μm and 150 μm, for example 50 μm.

We also describe an aerosol generator comprising a core of any example, and a power conductor for delivering electrical power to the vibration drive. In one example, the power conductor comprises a spring pin contacting a first surface and another spring pin contacting an opposed surface of the vibration drive.

In one example, the vibration drive comprises a discrete drive element in one example a piezoelectric drive element. In some examples, the power conductor comprises spring pins. The power conductor may comprise a spring pin contacting a first surface and another spring pin contacting an opposed surface via a terminal which extends around and encompasses the core around a side edge.

In some examples, the terminal has a first limb engaged by a spring pin and pressing against an insulator on the core, and a second limb which provides power to a side of the vibration drive. In some examples, the spring pins are parallel to each other and are both on an upstream side or a downstream side of the core. In some examples, the vibration drive comprises a discrete drive element, in one example a piezoelectric drive element. In some examples, the discrete drive element is seated in a groove in the support.

### BRIEF DESCRIPTION OF THE DRAWINGS

The invention will be more clearly understood from the following description of some embodiments thereof, given by way of example only with reference to the accompanying drawings in which:

FIG. 1 is a sectional side view of an aerosol generator core of one example, whereby an integral perforated membrane and support structure is integrally fabricated from a material that exhibits intrinsic piezoelectric properties and vibrates by direct application of an electrical drive signal and does not require the assistance of an external separate discrete vibration drive element;

FIG. 2 is a side sectional view of another example, in which the perforated membrane is planar and has the same thickness as the surrounding annular support, and FIG. 3 is a similar view of an example in which the perforated membrane is planar and with a smaller thickness than the surrounding support;

FIG. 4 is a sectional side view of an aerosol generator having an integral core and power provided by conducting spring pins and a conducting via through the support;

FIG. 5 is a sectional side view of another example of an aerosol generator having an integral core and power provided by conducting spring pins and a discrete conducting wraparound element around the support providing electrical connections from one side to an opposing conductor on the opposite side of the support;

FIG. 6 is a sectional side view of a further aerosol generator core with a body which in an integrated manner provides the perforated membrane, the support, and the vibration drive, and in this case power conducting terminals engage electrodes on the surface of the body in diametrically opposed locations;

FIG. 7 is a diagrammatic sectional side view of an alternative aerosol generator of the invention with a discrete vibration drive element embedded within the support structure and with power provided by pins protruding from the structure;

FIG. 8 is a diagrammatic sectional side view of an alternative aerosol generator of the invention with a discrete vibration drive element mounted exterior to the vibrating membrane structure, and with power provided by spring pins in physical contact with the side of the membrane opposite that upon which the vibration element is mounted;

FIG. 9 is diagrammatic sectional view showing an alternative aerosol generator with a discrete vibration drive element mounted exterior to the vibrating membrane structure, and with power provided by spring pins in physical contact with the same side of the membrane as that upon which the vibration element is mounted; and

FIG. 10 is diagrammatic sectional view showing an alternative aerosol generator with discrete vibration drive element mounted exterior but recessed into the vibrating membrane structure, and with power provided by spring pins in physical contact with the same side of the membrane as that upon which the vibration element is mounted.

### DETAILED DESCRIPTION

The invention provides aerosol generator cores and aerosol generators which are of simpler construction than is known in the field. In this specification the term “core” means the membrane and its support at least. In some examples the core encompasses a membrane and a support in an integrated manner, the combined membrane and support having generally the same footprint as a known aperture plate attached to a washer-shaped support. The core may in some examples also incorporate in an integrated manner a vibration drive, for example the core body being of piezoelectric material. In the latter example the core is preferably of a composite material, having required strength, vibration, and piezoelectric performance characteristics.

Due to the integrated architecture of the core, the core is simpler than heretofore and avoids problems of matching of tolerances and materials of components and the attachment manufacturing steps, such as attachment of an aperture plate to a washer-shaped support.

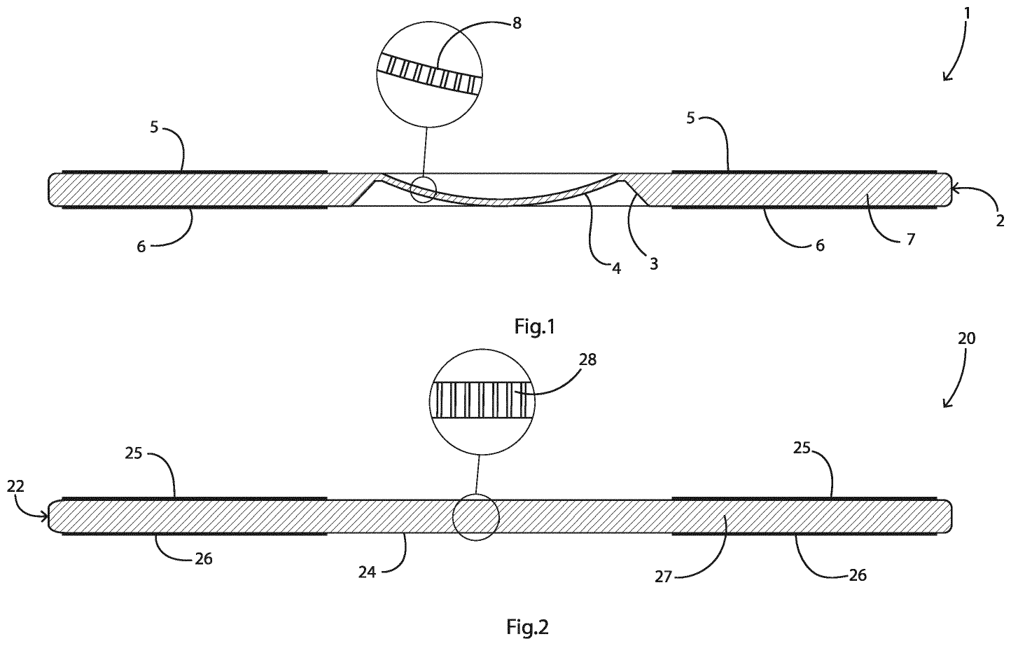

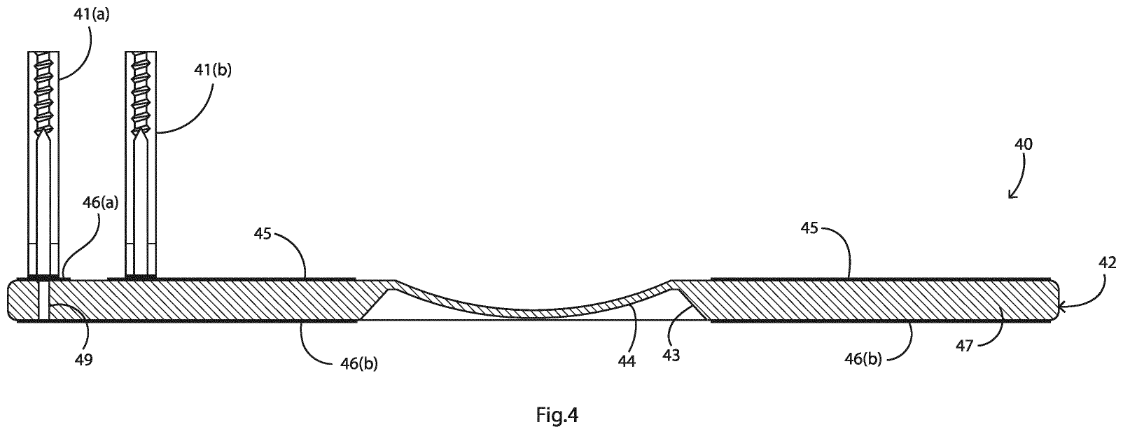

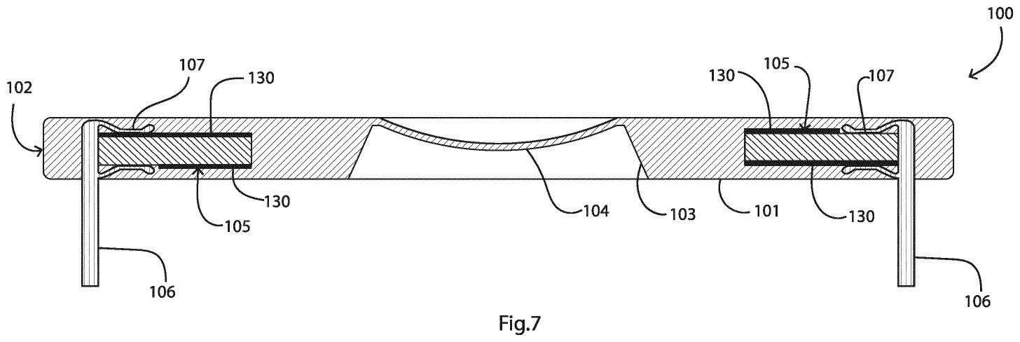

In the following description there are various embodiments, grouped with the first (FIGS. 1 to 6) group having the membrane, the support, and the vibration drive being fully integrated, and a second group (FIGS. 7 to 10) in which the membrane and the support are integrated and there is a discrete vibration drive. In the latter case the discrete drive element may be the sole vibration drive or it may augment the inherent vibration drive characteristic of the body. The electrode and terminal features of the various examples are not limited to those examples and may be applied to different examples. For example, FIG. 4 shows the terminals including a plated through hole for conducting power to the outlet-side electrode for power to the body with inherent vibration drive characteristics, however a plated through hole may be used to provide power to a surface of a discrete drive element. In the latter example the pins may be on the inlet side, the discrete drive element on the outlet side, and the plated through hole delivers power to the inlet-facing surface of the discrete vibration drive element where it is attached to the support and a clip delivers power to its (downstream facing) opposing surface.

### Aerosol Generator Core with Integral Membrane, Support, and Vibration Drive

Referring to FIG. 1 a nebulizer aerosol generator core 1 comprises a main body 2 which is shaped in the form of a washer-shaped annular support 7 and an integral dome-shaped perforated membrane 4 shaped to provide an aerosol-side recess 3. The dome shape of the membrane 4 provides an inlet side concave shape for retention of liquid to be aerosolized. There are annular electrodes 5 and 6 on the inlet and outlet sides respectively, adhered to the annular support 7. Apertures in the perforated membrane as shown diagrammatically in an enlarged view of part of the membrane. They may be of straight cylindrical shape or curved, depending on the use requirements.

The electrodes 5 and 6 are created using sputtering in one example. Sputtered silver, gold, aluminium or copper may be used with a nickel protection layer on the copper to prevent oxidation of the copper surface. Alternatively, Indium Tin Oxide (ITO) may be applied for increased electrical conductivity and chemical resistance and transparency. ITO may also be applied by sputtering or using physical vapour deposition of electron beam evaporation techniques.

In another example the electrodes are deposited by screen printing.

Sputtered electrode thickness is typically in the range 10-100 nm while thicker electrodes in the range 1-10 μm may be applied using screen printing. Most commonly, silver is the metal of choice for screen printing.

Electrode patterning can be created additively during the application process for example when screen printing or may be a subtractive process. Subtractive etch processes may be chemical or mechanical in nature. Ferric Chloride etching may be used for removal of sputtered copper; Hydrochloric Acid (HCL), Potassium Hydroxide (KOH) or Sodium Hydroxide (NaOH) may be used for removal of sputtered Aluminium; Dilute HCL may be used for removal of sputtered ITO and thick printed silver electrodes can be carefully removed by acetone. Sputtered Cu/Al/Gold can be removed by ultrafast UV laser (nanosecond or faster to avoid heat transfer to the underlying substrate). Dry film photolithography may also be used.

The material of the support 7 is a piezoelectric polymer so that when an electric drive is applied to the electrodes 5 and 6 it vibrates and transfers the vibrations to the membrane 4, in addition to performing the function of a physical support for the membrane 4. The material of the core body 2 is a composite construct fabricated in a single process step and which exhibits intrinsic piezoelectric properties.

The electrical power may be provided by conducting terminals such as pins or leaf springs, or clamps, depending on the preferred requirements for the physical arrangement of the aerosol generator.

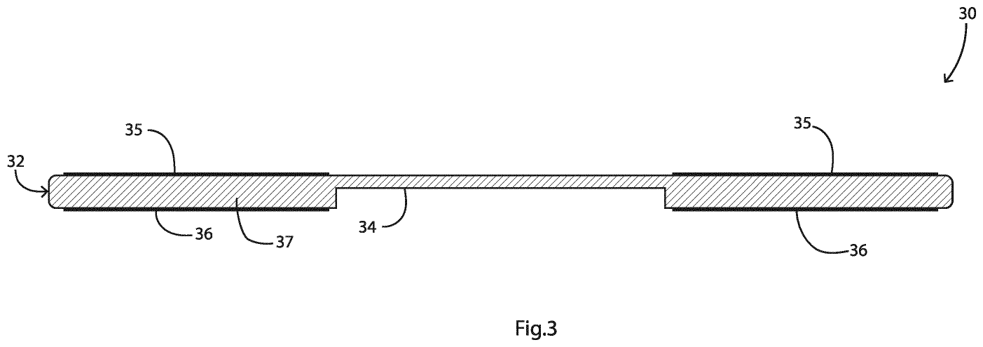

FIG. 2 shows a core 20 having an integral body 22 which has an overall disc shape, providing a support 27 and an integral perforated membrane 24 which has the same thickness as the surrounding support. Inlet side and outlet side electrodes 25 and 26 deliver power to the inlet and outlet sides of the body 22 to cause it to vibrate.

FIGS. 1 and 2 illustrate an enlarged cross-sectional view to show diagrammatically the aerosol-forming apertures, not shown to scale. The domed perforated membrane 4 is shown with apertures 8 and the perforated membrane 24 is shown with apertures 28. These apertures have a size in the range of 1 to 10 μm, so that upon vibration of the core body 2, 22, the membrane 4, 24 displaces at high frequency causing the liquid on the inlet (top) side to pass through the apertures and become aerosolised with droplets having a size in the range of 1 to 10 μm.

The perforated membrane 4 is domed, whereas the membrane 24 is co-planar with the body 27. In other examples the membrane has the same thickness as the body (as the membrane 24 has) but is domed. Also, the membrane 4, which has a smaller thickness than the surrounding body, has a rim at the level of the inlet side of the body 7. However, it is envisaged that in other examples a membrane which is thinner than the body may be located closer to the outlet side, for example with a convex surface extending distally (in the outlet direction) of the outlet side of the body. Irrespective of whether the membrane is flat (planar) or domed it may be at any suitable axial position relative to a longitudinal axis through the core. It will be apparent from the discussion below regarding manufacturing techniques that there is excellent versatility in choice of membrane shape and axial position. This is a major benefit of the support and membrane being integral.

FIG. 3 shows a core 30 having an integral body 32 which has an overall disc shape, providing a support and an integrated perforated membrane 44 which has a smaller thickness than the surrounding support 37. Inlet side and outlet side electrodes 35 and 36 deliver power to the inlet and outlet sides of the body 32 to cause it to vibrate. The membrane 34 planar and its surface forms a continuum with the inlet side surface of the body 37. However, in other examples it is further downstream in the axial dimension, for example to form part of a liquid reservoir well over the membrane.

FIG. 4 shows a core 40 having an integral body 42 which has an overall disc shape, providing a support 47 and an integral perforated membrane 44 which is dome shaped and has a similar configuration to the membrane 4. There is a recess on the outlet side formed by a shoulder 43. Inlet side and outlet side electrodes 45 and 46 deliver power to the inlet and outlet sides of the body 42 to cause it to vibrate. The inlet side electrode 45 is annular but does not extend radially to the full outer edge of the body 42. The annular space around the electrode 45 has an annular electrode island portion 46(a) which is isolated from the top side of the body 47 but is linked electrically by a conducting via 49 which has an insulating sleeve, to an outlet side annular electrode 46(b). The electrode portions 46(a) and 46(b) together form the outlet side electrode 46, both portions being electrically isolated from the inlet side electrode 45. Conducting spring pins 41(a) and 41(b) deliver power to the electrodes 46 and 45 respectively.

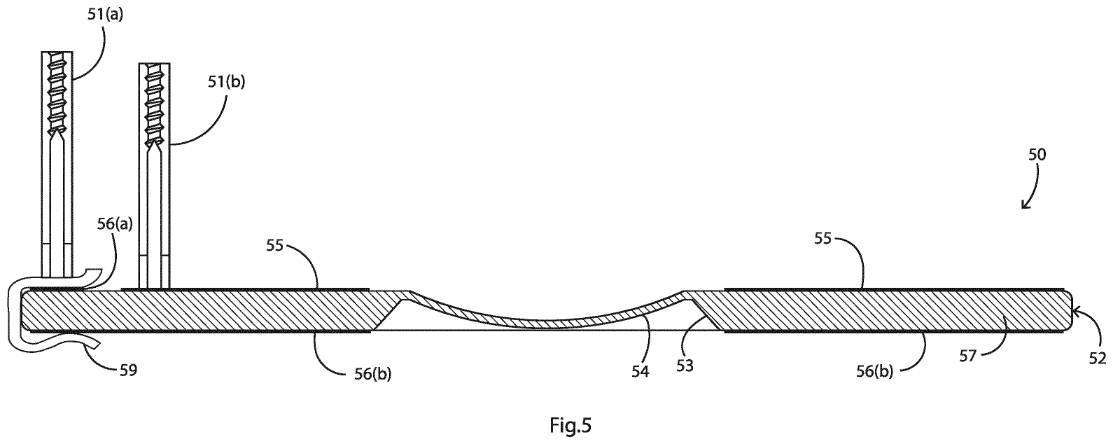

FIG. 5 shows a core 50 having an integral body 52 which has an overall disc shape, providing a support 57 and an integral perforated membrane 54 which is dome shaped and has a similar configuration to the membrane 4. There is a recess 53 on the outlet side of the membrane 54. Inlet side and outlet side electrodes 55 and 56 are provided to deliver power to the inlet and outlet sides of the body 52 to cause it to vibrate. The inlet side electrode 55 is annular but does not extend radially to the full outer edge of the body 52. The annular space around the electrode 55 has an annular electrode island portion 56(a) which is isolated for the top side of the body 52 but is linked electrically by a radial clamp or clip 59 to an outlet side annular electrode 56(b). The electrode portions 56(a) and 56(b) are electrically isolated from the inlet side electrode 55. Conducting spring pins 51(a) and 51(b) deliver power to the electrodes 56 and 55 respectively.

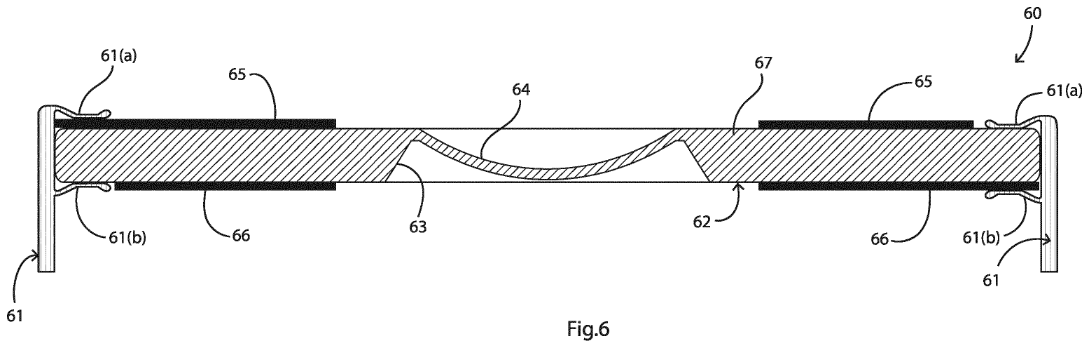

In another example shown in FIG. 6, a core 60 has a body 62 of piezoelectric material with a support 67, and an integral perforated membrane 65 over a recess formed by a shoulder 63. The vibration drive is provided by the annular core body support 67, which is fabricated from a material exhibiting piezoelectric properties. In this example, the metallised electrodes 65 and 66 of the piezoelectric body 62 are contacted by opposed spring clip terminals 61 having terminal pins in an embedded lead frame arrangement. The terminals 61 have radial springs 61(a) and 61(b) engaging opposed sides of the core in a clamping-type arrangement on opposed sides. In this example the top and bottom electrodes 65 and 66 are offset so that power is conducted to the inlet-side electrode 65 by the left side terminal 61 and to the outlet-side electrode 66 by the right-hand terminal 61.

The dimensions of the cores 1, 20, 30, 40, 50, and 60 are, for various examples:
Full diameter, between 5 mm and 50 mm, preferably between 10 mm and 25 mm, for example 15 mm.
Membrane 4 diameter, between 1 mm and 50 mm, preferably between 2 mm and 10 mm diameter, for example 4 mm.
Body thickness, between 20 μm and 5000 μm, preferably between 30 μm and 1500 μm, for example 500 μm.
Perforated Membrane thickness, between 10 μm and 300 μm, preferably between 30 μm and 150 μm, for example 50 μm.
Aerosol forming apertures in the membrane, 1 μm to 10 μm, preferably between 2 μm to 5 μm.

### Manufacture of the Integral Core Body

The membranes 4, 44, 54, and 64 each have an integrated dome profile. The membranes 24 and 34 are examples of membranes which are flat. The apertures within the membrane can either be created as part of the substrate manufacturing process or post-formed into the membrane area, by using for example micromachining processes such as laser drilling. In various examples the shape is formed by hot embossing, moulding, inkjet, or 3D Printing or UV Nanoimprint Lithography.

These processes were chosen due to them having the potential of creating complex and intricate geometries down to the micron scale. The speed at which these processes can create a product are not taken into consideration. The process is not limited to a single step. If required, multiple processes can be used in conjunction to achieve the desired result.

Hot embossing is a process which relies on increasing the temperature of a polymer sheet above its melting range followed by pressing a heated mould into the polymer generating pressure to fill the surface structures. In hot embossing, a mould is attached to one clamp, and the polymer material or substrate is attached to another clamp, both clamps are heated, one clamp is stationary while the other lowers slowly and gradually as the polymer slowly deforms around the mould.

When it is lowered, the polymer is softened and forms into the shape of the mould. The process takes between 5-30 mins to complete. The applications of hot embossing can be found in optical devices such as compact discs, lenses, mirrors, optical benches, wave guides and switches (Omar 2013) and (Becker & Heim, 1999). Common materials used in Hot Embossing are Polycarbonate (PC), Cyclic Olefin Copolymers (COC) and Polymethylmethacrylate (PMMA).

Micro injection is very similar to standard injection moulding, but on a micro-scale. Similar to standard injection moulding, polymer granules are fed into the hopper, are pushed along the barrel where they are softened and molten, and then injected into a mould under pressure where it solidifies. (Surace, et al., 2012) and (Omar, 2013).

Manufacture of the core may alternatively be by way of 3D printing. There are many 3D printing techniques, all with varying production speeds, feedstock material choices and resolution capabilities. Basic FDM (Fused Deposition Modelling) 3D printing adds layers on top of layers through a hot nozzle (called the extruder) of a set diameter to produce the 3D object (O'Neal, 2019). Advanced printers using lasers on photosensitive materials, such as Multiphoton Lithography can have high resolution (˜250 nm) and can produce parts down to the micrometre scale. (Prabhakaran, 2019).

Another manufacturing approach is Digital Light Processing (DLP) and Stereo Lithography (SLA), both methods used in 3D printing with resin baths, and both use UV light on photosensitive resin to harden it. In both printing methods, a platform is moved vertically as layer by layer is being solidified by the laser/projector. A DLP printer can print faster compared to SLA, therefore it has the ability to solidify an entire plane at a time, compared to SLA which is essentially drawing the objects layer by layer with a singular laser. The accuracy and resolution can also vary between the two processes. In DLP printing, square pixels are applied in a combination to solidify the resin which causes the 3D object to have steps around the edges. However, this process is being refined and anti-aliasing techniques are being applied with a specific kind of DLP printing called CDLM DLP printing. The anti-aliasing function essentially smoothens the edges of the subject removing the steps. Any photopolymer can be used in either of these two processes, therefore theoretically if a photo-initiator is introduced into a polymer, it can be used as the resin for SLA/DLP printing (Waheed, et al., 2016)

Nanoimprint Lithography (NIL) can create sub-micron features on a polymer surface such as a diaphragm. It creates a pattern via deformation onto a resist material using a mould. There are many types of lithography, but two fundamental types are Thermal Nanoimprint Lithography (T-NIL) and UV Nanoimprint Lithography (UV-NIL) with both being capable of creating features as small as 10 nm. The main differences between T-NIL and hot embossing are that hot embossing is slower and can work on larger mould structures. NIL applications mainly cover nanoelectronics, nano-optoelectronics, nanophotonic, nano-biology, optical components, etc. (Omar, 2013).

Table 1 is a summary comparison of the various key process parameters for some of the techniques described above.

TABLE 1
Comparison of replication processes. (Omar, 2013)

| Criteria | Injection Moulding | Hot Embossing | UV-Imprinting |
|---|---|---|---|
| Cycle Time | 1 to 10 Seconds | 5-30 min | 10-30 sec |
| Flow Length | Length of Runner | Height of Cavity | Height of Cavity |
| | System | | |
| Raw Material | Granules | Sheets | Thin Film |
| Materials | Thermoplastic | Thermoplastics, | UV-Curable resist |
| | Polymers, Ceramics | glass and metals | |
| | and metals | | |
| Residual Stress | High | Medium | Low |
| Moulding Window | Above melting | Above Tg or in the | Room Temperature |
| | Temperature | melting range | |
| Serial Production | Large | Medium | Medium |

The aerosol forming apertures of the perforated membranes 4, 24, 34, 44, and 54 may be formed by laser machining. Laser machining of apertures with appropriate geometries for aerosol formation is possible on many common engineering polymers including but not limited to—polyimide (PI), polycarbonate (PC), polyester (PET), PEEK, Nylon, PMMA, PTFE, FEP and acrylics. All these polymers can be cut with high precision and high quality with appropriate selection of laser type and laser parameters. This is known on commercially available nebulisers using membranes fabricated primarily with Polyimide materials.

Laser machining of PVDF for the production of sensors and actuators is well known in the art. Capineri et al [7] demonstrated laser ablation of PVDF film with negligible effects on the uniformity of the pyroelectric response. Chung et al [8] and Wang et al [9] applied excimer lasers to pattern electrodes on 25 μm thick PVDF film. Farlow et al used a frequency-doubled copper vapor laser to cut miniature cantilevers in PVDF film and Fu et al fabricated a cantilever beam using PVDF film with a Nd: YAG laser.

When it comes to precision micromachining for providing apertures of size lum to 10 μm required to create small droplets for inhalation, highly sophisticated laser systems are required. Femtosecond lasers offer the versatility to micromachine almost any material with high precision and minimum damage. Conventional laser micromachining uses pulses of laser energy longer than several tens of picoseconds. Conversely femtosecond pulses deposit the laser energy into the electrons of the material on a time scale much shorter than the transfer time of this energy to the bulk of the material. Zhao et. al. showed how the resultant reduction in thermal diffusion leads to an improvement of the process efficiency and a significant reduction of material melting that reduces process precision and efficiency. Stoian et. al demonstrated that laser ablation of dielectrics was possible with temporally shaped femtosecond pulses. Lee et. al. demonstrated selective removal and patterning of NiCu coating from PVDF film and ablation characteristics of the PVDF film using a femtosecond laser. Process parameters such as laser fluence, feed rate and number of machining passes was shown to have an impact on the quality and accuracy of material removal.

The core electrodes apply a voltage across the core from an alternating voltage source providing a voltage in the range 1 Volt peak-to-peak to 3000 Volts peak-to-peak, more typically in the range 100 Volts to 1500 Volts peak-to-peak. The electrodes 5, 6, 25, 26, 35, 36, 45, 46, 55, and 56 may be thin metal bodies, as described above. In other examples the electrodes may take the form of a metallisation of the top and bottom surfaces of the polymer body.

### Electrical Drive Aspects

The frequency of the voltage can be in the range 500 Hz to 5000 Khz, more typically in the range of 1 Khz to 500 Khz. It is advantageous that the applied voltage and frequency are sufficient to achieve a membrane displacement of at least lum. The piezoelectric effect with the above drive voltages and frequencies establishes modes of vibration within the membrane with peak displacement ranging from 0.1 μm to 100 μm, more typically in the range 0.5 μm and 10 μm. Optimum drive frequency for best aerosol performance (highest nebulisation output rate with lowest droplet size) is achieved when the drive frequency is closely matched with the natural resonant frequency of the system in the wet state. Furthermore, an increase in drive voltage creates a strong corresponding increase in nebulisation output rate with typical drive voltages in the range 50-100 Volts peak-to-peak for discrete vibrating element piezoelectric Lead Zirconium Titanate (PZT) driven devices. The voltages applied result in sufficient displacement (>1.0 μm) of coupled PZT driven vibrating mesh membranes such that droplet release results. Since the piezoelectric constant of PVDF is approximately a factor of 10× lower than that of PZT, a voltage peak-to-peak 5-20× larger will be required to create comparable displacements. Therefore, the voltages required for PVDF devices may be required to be of the order of 1000 Volts peak-to-peak or greater.

### Material of the Integral Core

The material of the core 1 comprises a piezoelectric polymer, in one preferred example Polyvinylidene Difluoride (PVDF), which is a highly non-reactive thermoplastic fluoropolymer produced by the polymerization of vinylidene difluoride. It is known that Nylon and PVC can exhibit piezoelectric properties, however they are not as highly piezoelectric as PVDF and its copolymers. Like some other ferroelectric materials, PVDF is also pyroelectric, producing electrical charge in response to a change in temperature. PVDF strongly absorbs infrared energy in the 7-20 μm wavelengths covering the same wavelength spectrum as heat from the human body.

The PVDF membrane is thin and flexible with low density, low impedance, and excellent sensitivity. The PVDF material of the cores 1, 20, 30, 40, 50 is chemically inert and biocompatible. Unlike some other piezoelectric materials, such as lead zirconate titanate (PZT), PVDF has a negative d33 value. Physically, this means that PVDF will compress instead of expanding or vice versa when exposed to the same electric field.

PVDF properties include:
Wide frequency range (0.001 Hz to 1 GHz)
High dynamic range (10⁻⁸ to 10⁶ psi)
Low acoustic impedance-close match to water, human tissue, and adhesive systems
High elastic compliance
High voltage output—10 times higher than piezo ceramics for the same force input
High dielectric strength—withstanding strong fields (75V/μm) where most piezo ceramics depolarize.
High mechanical strength and impact resistance (10⁹-10¹⁰ Pa modulus)
High stability—resisting moisture (<0.02% moisture absorption), most chemicals, oxidants, and intense ultraviolet and nuclear radiation
Can be fabricated into unusual designs
Can be glued with commercial adhesive

One major advantage of piezoelectric polymer over piezoceramic is its low acoustic impedance which is closer to that of water, human tissue, and other organic materials. For example, the acoustic impedance of piezoelectric polymer is only 2.6 times that of water, whereas piezo ceramics are typically 11 times greater. A close impedance match permits more efficient transduction of acoustic signals in water and tissue. Piezoelectric polymer has low density and excellent sensitivity and is mechanically tough. The compliance of piezoelectric polymer is 10 times greater than the compliance of ceramics. Piezoelectric polymer is well suited to strain sensing applications requiring very wide bandwidth and high sensitivity. As an actuator, the polymer's low acoustic impedance permits the efficient transfer of energy into air and other gases. Table 2 lists typical properties of commercially available piezoelectric PVDF polymers. Table 3 provides a comparison of the piezoelectric properties of PVDF polymer and two popular piezoelectric ceramic materials. (Piezo Film Sensors Technical Manual https://mma.pages.tufts.edu/emid/piezo.pdf)

TABLE 2
Typical Properties of piezoelectric polymer

| Symbol | Parameter | | PVDF | Copolymer | Units |
|---|---|---|---|---|---|
| t | Thickness | | 9, 28, 52, 110 | <1 to 1200 | μm (micron) |
| d₃₁ d₃₃ | Piezo Strain constant | | 23 −33 | 11 −38 | $1 {0}^{- 12} \qquad \frac{m / m}{V / m} or \frac{C / {m}^{2}}{N / {m}^{2}}$ |
| g₃₁ g₃₃ | Piezo strain constant | | 216 −330 | 162 −542 | $1 {0}^{- 3} \qquad \frac{V / m}{N / {m}^{2}} or \frac{m / m}{C / {m}^{2}}$ |
| k₃₁ | Electromechanical Coupling | | 12% | 20% | |
| k_{f} | Factor | | 14% | 25-29% | |
| C | Capacitance | | 380 for 28 μm | 68 for 100 μm | pF/cm² @ 1 KHz |
| Y | Young's Modulus | | 2-4 | 3-5 | 10⁹ N/m² |
| V₀ | Speed of | Stretch Thickness: | 1.5 | 2.3 | 10³ m/s |
| | sound | | 2.2 | 2.4 | |

| p | Pyroelectric Coefficient | 30 | | 40 | | 10⁻⁶ C/m² ° K. |
|---|---|---|---|---|---|---|
| ε | Permittivity | 106-113 | | 65-75 | | 10⁻¹² F/m |
| ε/ε₀ | Relative Permittivity | 12-13 | | 7-8 | | |
| ρₘ | Mass Density | 1.78 | | 1.82 | | 10⁵ kg/m |
| ρₑ | Volume Resistivity | >10¹⁵ | | >10¹⁴ | | Ohm Meter |
| R | Surface Metallization | <3.0 | | <3.0 | | Ohms/square for NiAl |
| R | Resistivity | <0.1 | | 0.1 | | Ohms/square for Ag Ink |
| tan δₑ | Loss Tangent | 0.02 | | 0.015 | | @1 KHz |
| | Yield Strength | 45-55 | | 20-30 | | 10⁶ N/m² (stretch axis) |
| | Temperature Range | −40 to | | −40 to | | ° C. |
| | | 80 . . . 100 | | 115 . . . 145 | | |
| | Water Absorption | <0.02 | | <0.02 | | % H₂O |
| | Maximum Operating Voltage | 750 | (30) | 750 | (30) | V/mil (V/μm), DC, @ 25° C. |
| | Breakdown Voltage | 2000 | (80) | 2000 | (80) | V/mil (V/μm), DC, @ 25° C. |

TABLE 3
Comparison of Piezoelectric Materials

| Property | Units | PVDF Film | PZT | BaTi0₃ |
|---|---|---|---|---|
| Density | 10³ kg/m³ | 1.78 | 7.5 | 5.7 |
| Relative Permittivity | ε/ε₀ | 12 | 1,200 | 1,700 |
| d₃₁ Constant | (10⁻¹²) C/N | 23 | 110 | 78 |
| g₃₁ Constant | (10⁻³) Vm/N | 216 | 10 | 5 |
| k₃₁ Constant | % at 1 KHz | 12 | 30 | 21 |
| Acoustic Impedance | (10⁶) kg/m²-sec | 2.7 | 30 | 30 |

Copolymers of PVDF permit use at higher temperatures (135° C.) than standard PVDF. Thickness extremes are possible with copolymers that cannot be readily attained with PVDF. These include ultrathin (200 Å) spin-cast coatings that enable new sensor-on-silicon applications, and cylinders with wall thicknesses in excess of 1200 μm for sonar applications.

Micro-opto-electro-mechanical systems utilize the advantages offered by smart materials, combining their sensing and actuation capabilities. A piezoelectric polymer film is one such smart material with unique sensing/actuation capabilities. It produces a voltage in response to an applied compressive or tensile mechanical stress or strain, making it an ideal dynamic strain gage. The piezoelectric polymer's compliance is about ten times higher than other piezoelectric materials such as ceramic and quartz. Conversely, the piezoelectric polymer film undergoes a proportional change in dimension under the influence of an applied electric field.

Another example of the material for the integral core is P(VDF-trifluoroethylene), or P(VDF-TFE), available in ratios of about 50:50 and 65:35 by mass (equivalent to about 56:44 and 70:30 molar fractions). These copolymers improve the piezoelectric response by improving the crystallinity of the material. While the copolymers' unit structures are less polar than that of pure PVDF, the copolymers typically have a much higher crystallinity. This results in a larger piezoelectric response: d33 values for P(VDF-TFE) have been recorded to be as high as −38 pC/N.

In one example the material is of the type described in [1], which describes the use of (P(VDF-TrFE) in piezoelectric actuator applications.

In another example the material may be P(VDF-TrFE) supplied by Piezotech™, fabricated with specific piezoelectric properties (see https://www.piezotech.eu/en/).

In another example, the material comprises Piezoelectric PVDF supplied by PolyK Technologies, LLC (see https://piezopvdf.com/). Such material may be uniaxially or biaxially oriented with high piezoelectric response.

In another example, the material comprises Piezoelectric PVDF supplied by TE Connectivity (see https://www.te.com/usa-en/home.htm)

These materials advantageously have the structural attributes for withstanding the high-frequency vibratory environment of an aerosol generator which has been shown viable for cyclic operation, see for example [1].

In various examples the material comprises a composite of polymers selected from Polyamide, (PA), Polyimide (PI), Polycarbonate (PC), Polyether-ether-ketone (PEEK), Polyethylene-terephthalate (PET) or Acrylonitrile-butadiene-styrene (ABS) families of polymers with a material that exhibits piezoelectric behaviour such as Lead Zirconium Titanate (PZT).

The following are references which each describes a material which would be suitable to provide a composite core which is piezoelectric. These documents are incorporated herein by reference.

### Examples with Discrete Vibration Drive

FIGS. 7 to 10 illustrate other embodiments with discrete vibration drives and power conductors for delivering power to them. The discrete vibration drives in these examples may be the sole vibration drive or may be provided to augment a vibration drive capability of the support, having a material of any of the support bodies 2, 22, 32, 42, 52, or 62 described above, and also similar power conductors and including surface electrodes and conductive pins or springs.

Any of these power conductors may be employed to deliver power to an integrated vibration drive such as those of FIGS. 1 to 6. The core body may have electrode surfaces which are suited to engagement with spring contacts, whether they are pins or cantilevered, or bent in a shape such as a C-shape or a ridge.

As for the embodiments of FIGS. 1 to 6 the aerosol generator core has the advantage of being provided as a package which is self-contained with the functions of an integrated support body and perforated membrane, and the body having the vibration drive within or adhered to the body as an overall discrete package.

Referring to FIG. 7 a core 100 with dimensions within the above ranges comprises a body 102 comprising a thermoset or thermoplastic polymer material (not piezoelectric), and an integral recess 103 spanned by an integral membrane 104. In this case the vibration drive is provided by a discrete piezoelectric actuator 105 which is annular and is completely embedded within the support 101 of the body 102 and is contacted by opposed spring clip terminals 107 linked with terminal pins 106 in an embedded lead frame arrangement. The terminals 107 engage electrodes 130 on the surfaces of the vibration element 105, and the electrodes 130 may be provided in any of the examples described above with reference to FIGS. 1 to 6. The material of the body 101 comprises in preferred examples polymers selected from Polyamide, (PA), Polyimide (PI), Polycarbonate (PC), Polyether-ether-ketone (PEEK), Polyethylene-terephthalate (PET) or Acrylonitrile-butadiene-styrene (ABS) families of polymers.

Similar fabrication techniques as were used in the embodiments of FIGS. 1 to 6 may be used for the body 101 and can be employed for both piezoelectric and non-piezoelectric polymers.

Additionally, the core body may be fabricated using conventional integrated circuit packaging and encapsulation techniques such as transfer moulding using thermosetting epoxy resin or injection moulding of thermoplastics such as Liquid Crystal Polymer (LCP), Polyetheretherketone (PEEK), Polyphtalamide (PPA) and Polyphenylene Sulfide (PPS).

The piezoelectric element is driven from an alternating voltage source establishing a voltage across the piezoelectric element in the range 1 Volt peak-to-peak to 1000 Volts peak-to-peak, more typically in the range 10 Volts to 300 Volts peak-to-peak. The frequency of the voltage can be in the range 500 Hz to 5000 kHz, more typically in the range of 1 kHz to 500 kHz.

The aerosol generator core may in other examples have a power conductor comprising a spring pin contacting a first surface and another spring pin contacting an opposed surface via a terminal which extends around and encompasses the core around a side edge. Such a terminal may have a first limb pressed by the spring pin against an insulator on the core, and a second limb which provides power to the core on the opposed side. This allows the spring pins to be parallel to each other, both on an upstream side or a downstream side of the core

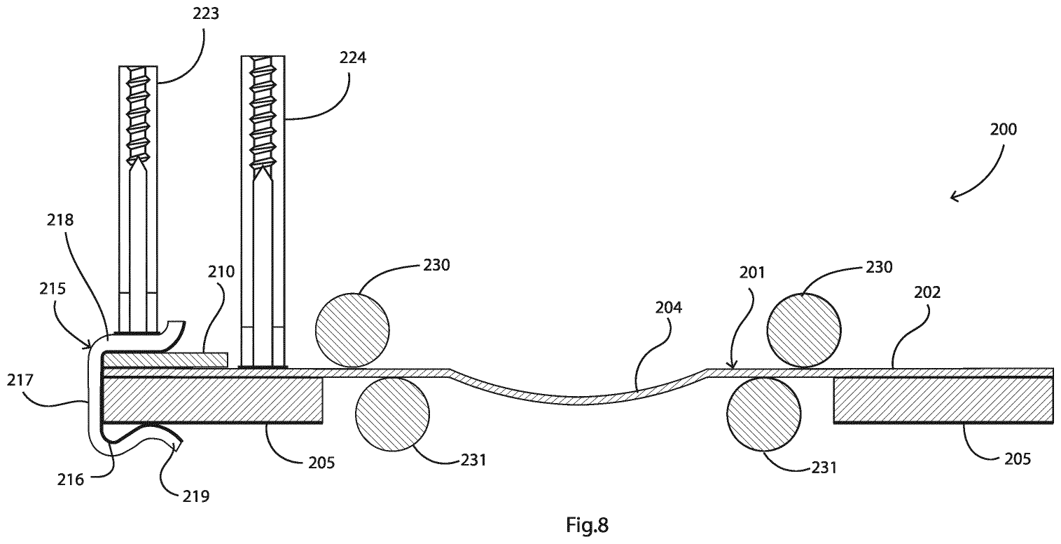

Referring to FIG. 8, in another example an aerosol generator 200 for a nebulizer comprises an integral support and membrane core 201 having a support portion 202 and a membrane 204. The material of the core body 202 is metallic in nature (for example Palladium-Nickel, Stainless Steel, or Hastelloy). In other examples it may be a metallised polymer.

The core 201 is supported by upper and lower O-ring seals 230 and 231 respectively, although any other suitable shape of resilient seal may be used. Such seals may be used in any of the cores described in the is specification. A piezoelectric annular element 205 is adhered to the underside of the core 201 around its outer edge, and on the opposed face of the core 201 there is an insulating annular ring 210. Power is provided by a pair of conducting spring pins 223 and 224, both on the top (upstream) side of the core and being parallel to each other. One pin 224 provides power to the top surface of the core body 202 for conduction of current through the body 202 to the top surface of the piezo element 205. Power is provided to the opposed, lower, surface of the piezo element 205 by a conductive clip 215 having an internal electrically contacting surface 216 and having a central portion 217 supporting two inwardly sprung limbs 218 and 219 which act like jaws to engage the side edge of the aerosol generator. A pin 223 engages the top surface of the limb 218, and power is conducted around the central portion 217 and the lower limb 219 to engage the bottom surface of the piezo element 205. The insulating ring 210 and a coating around the core outer edge prevent electrical contact of the clip 215 other than with the bottom surface of the piezo element 205, the terminal 215 extending around the outer edge to press together a sandwich of the insulator 210, the core body 202, and the piezo element 205. It will be noted that the insulator 210 is needed here because the body 201 is conductive itself. However, if it were not, power could be provided by wrap-around terminals and offset electrodes as shown in FIGS. 6 and 7.

In this example, power is provided to the vibration generator in a compact and simple manner, the material of the core itself not necessarily being piezoelectric.

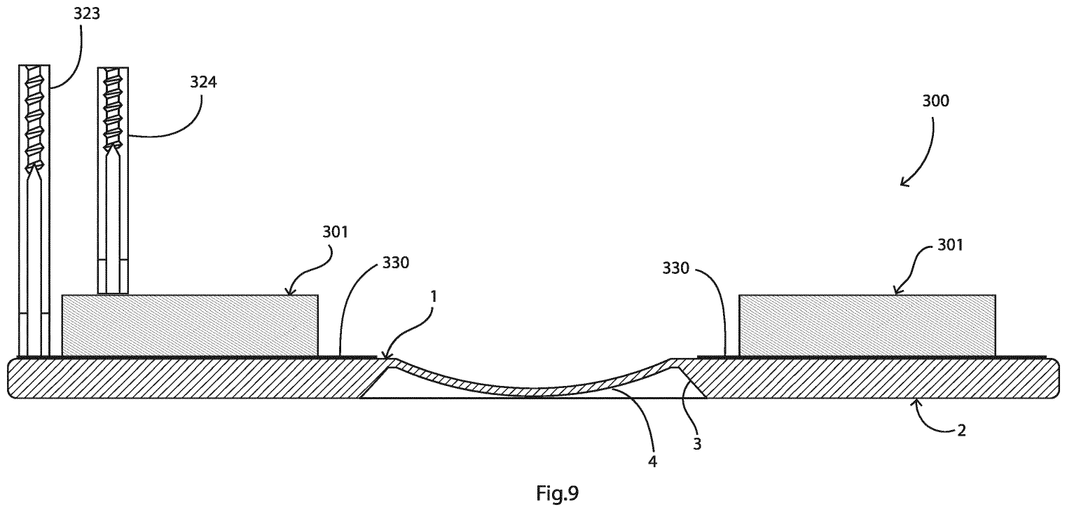

Referring to FIG. 9 an alternative aerosol generator 300 has the core 1 and also an additional drive provided by a discrete annular piezoelectric vibration element 301. Power is delivered by a spring pin 324 to the top surface of the element 301 (which has a thin electrode surface), and to the bottom surface of the element 301 via a spring pin 323 via a conductive film 330 on the body 2. Additionally, power is provided to the body 2 in a manner such as described in FIG. 4 or 5.

In other examples, the material of the core could be either a polymer or a metal. In the case of a polymer, it would need to have a conductive layer (for example, 330 in FIG. 9) applied on its top surface to enable conduction between spring pin and the bottom surface of the piezo. This conductive layer can be applied by sputtering, laser etching, or chemical methods as are well known in the art.

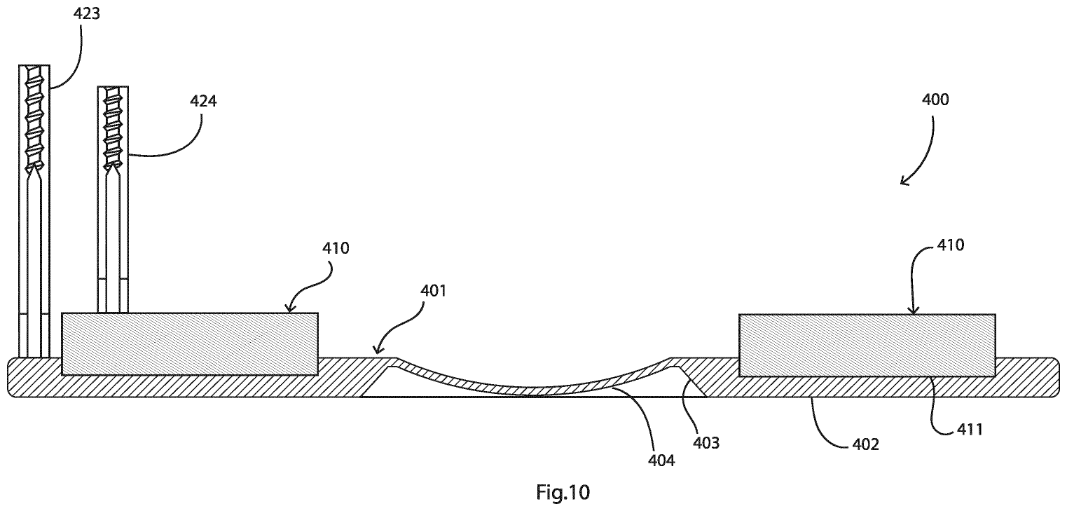

Referring to FIG. 10 an aerosol generator 400 has a core 401 with an integral support body 402 and membrane 404, and a recess shape 403. This body material is metallic in nature (for example Palladium-Nickel, Stainless Steel, or Hastelloy) and is not piezoelectric. The vibration drive is provided by an annular piezoelectric element 410 which is seated in an annular groove 411 in the top side of the core 401. Power is provided by a spring pin 423 to the core 401 and from there to the underside of the piezoelectric element 410, and by a spring-pin 424 directly to the top of the piezoelectric element 410.

Of course, in examples where the body is non-conducting, electrodes are required on its surface sufficient to deliver power from the pin 423 to the underneath of the element 410.

### Advantages

The invention provides a simple low-cost integrated aerosol generator core that is compatible with very high-volume production at low cost. It is envisaged that its manufacture would reduce cost, facilitating large scale production, and making the device available to many more patients is a shorter timeframe than is currently realisable with the existing technology.

Several advantages may be summarised as follows:
Scalable in very high volume
Simple assembly
Reduced device performance variation by minimising influence of multiple component and process tolerances
Adaptable/versatile aperture geometry to enable nebulisation of different drugs.
Highly corrosion-resistant compared with low-cost electroformed metal (e.g. Nickel)

### Other Alternatives

The invention is not limited to the embodiments described but may be varied in construction and detail. In various examples the core may have a discrete vibration drive on top with both connections made topside—one on the piezo and one on the support. Alternatively, there may be a discrete vibration drive on a bottom surface, possibly with wrapped-around or through hole via connection as described for example in FIG. 8. Alternatively, there may be a discrete vibration drive embedded within the core and pins connected to the drive protruding through the core to provide interconnection. In other examples, where the core has a material exhibiting intrinsic piezoelectric properties this could be a single material (like PVDF or PVDF Copolymers) which are intrinsically piezoelectric in nature or a composite material comprising a standard polymer (e.g. PI, PC, PEEK) loaded with a secondary material (e.g. PZT or similar) that would impart piezoelectric properties on the structure. Any of the features/components described for the examples may be used in different examples. For example, where a discrete vibration drive is provided the membrane may be planar (as in FIGS. 2 and 3 for example) rather than domed.

### REFERENCES

[1] Pabst, O., Perelaer, J., Beckert, E., Schubert, U. S., Eberhardt, R. and Tünnermann, A., 2013. All inkjet-printed piezoelectric polymer actuators: Characterization and applications for micropumps in lab-on-a-chip systems. Organic Electronics, 14(12), pp. 3423-3429.
[2] Ambrosy, A. and Holdik, K., 1984. Piezoelectric PVDF films as ultrasonic transducers. Journal of Physics E: Scientific Instruments, 17(10), p. 856.
[3] Fu, Y., Harvey, E. C., Ghantasala, M. K. and Spinks, G. M., 2005. Design, fabrication and testing of piezoelectric polymer PVDF microactuators. Smart materials and structures, 15(1), p.S141.
[4] Lopes, A. C., Gutiérrez, J. and Barandiarán, J. M., 2018. Direct fabrication of a 3D-shape film of polyvinylidene fluoride (PVDF) in the piezoelectric β-phase for sensor and actuator applications. European Polymer Journal, 99, pp. 111-116
[5] Ostaševičius, V., Milašauskaitė, I., Daukševičius, R., Baltrušaitis, V., Grigaliūnas, V. and Prosyčevas, I., 2010. Experimental characterization of material structure of piezoelectric PVDF polymer. Mechanics, 86(6), pp. 78-82.
[6] Rowland, H. D. and King, W. P., 2004. Polymer deformation and filling modes during microembossing. Journal of Micromechanics and Microengineering, 14(12), p. 1625.
[7] Becker, H. and Heim, U., 2000. Hot embossing as a method for the fabrication of polymer high aspect ratio structures. Sensors and Actuators A: Physical, 83(1-3), pp. 130-135.
[8] Omar, F., 2013. Hot embossing process parameters: Simulation and experimental studies (Doctoral dissertation, Cardiff University).
[9] Surace, R., Trotta, G., Bellantone, V. and Fassi, I., 2012. The micro injection moulding process for polymeric components manufacturing. Intechopen.
[10] Prabhakaran, D. P., 2019. Two-Photon Lithography System. [Online] Available at: https://www.l3dw.com/two-photon-lithography-setup/
[11] O'Neal, B., 2019. ABS: Researchers Test Temperature & Speed Settings in FDM 3D Printing. [Online]: Available at: https://3dprint.com/236571/abs-researchers-test-temperature-speed-parameters-3d-printing/
[12] Formlabs, n.d. SLA vs. DLP: Guide to Resin 3D Printers. [Online]: Available at: https://formlabs.com/eu/blog/resin-3d-printer-comparison-sla-vs-dlp/
[13] Waheed, S., Cabot, J. M., Macdonald, N. P., Lewis, T., Guijt, R. M., Paull, B. and Breadmore, M. C., 2016. 3D printed microfluidic devices: enablers and barriers. Lab on a Chip, 16(11), pp. 1993-2013.
[14] Capineri L, Masotti L, Mazzinghi P and Mazzoni M 2000 Fabrication of pyroelectric PVDF linear arrays for diagnostic systems of high power CO2 laser beams Proc. 5th Italian Conf. Sensors and Microsystems pp 411-5
[15] Chung C H, Lee Y C, Kuo S H and Chiu C L 2005 A novel back scattering ultrasound transducer for non-destructive material evaluation and defect inspection IEEE Ultrasonics Symp. pp 174-7
[16] Wang Y C, Huang C H, Lee Y C and Tsai H S 2006 Development of a PVDF sensor array for measurement of the impulsive pressure generated by cavitation bubble collapse Exp. Fluids 41 365-73
[17] Farlow R, Galbraith W, Kelly S and Hayward G 2001 Micromachined unimorphs and bimorphs IEEE Ultrasonics Symp. pp 913-6
[18] Fu Y, Muralidhar G, Erol H and Qin L 2005 Design and fabrication of a hybrid actuator Smart Mater. Struct.
[19] Zhao J, Huettner B and Menschig A 2001 Microablation with ultrashort laser pulses Opt. Laser Techol. 33 487-91
[20] Stoian R, Boyle M, Thoss A, Rosenfeld A, Kom G, Hertel I V and Campbell E E B 2002 Laser ablation of dielectrics with temporally shaped femtosecond pulses Appl. Phys. Lett. 80 353-5
[21] Lee, S., Bordatchev, E. V. and Zeman, M. J., 2008. Femtosecond laser micromachining of polyvinylidene fluoride (PVDF) based piezo films. Journal of Micromechanics and Microengineering, 18(4), p. 045011.
[22] Moon, S. H., Chang, K. H., Park, H. M., Park, B. J., Yoo, S. K. and Nam, K. C., 2021. Effects of Driving Frequency and Voltage on the Performances of Vibrating Mesh Nebulizers. Applied Sciences, 11(3), p. 1296.

1-38. (canceled)

39. An aerosol generator core comprising a body having a support supporting an integral membrane and a vibration drive adapted to be connected to an electrical voltage supply.

40. An aerosol generator core as claimed in claim 39, wherein the vibration drive comprises the body, the body being configured to vibrate with application of an electrical voltage across it.

41. An aerosol generator core as claimed in claim 40, wherein the body comprises a piezoelectric polymer.

42. An aerosol generator core as claimed in claim 41, wherein the piezoelectric polymer comprises polyvinylidene fluoride (PVDF).

43. An aerosol generator core as claimed in claim 42, wherein the body comprises a PVDF copolymer.

44. An aerosol generator core as claimed in claim 43, wherein the piezoelectric polymer comprises (P(VDF-TrFE).

45. An aerosol generator core as claimed in claim 41, wherein the piezoelectric polymer comprises P(VDF-TFE).

46. An aerosol generator core as claimed in claim 40, wherein the body comprises a composite material comprising a polymer loaded with a secondary material that imparts piezoelectric properties on the body.

47. An aerosol generator core as claimed in claim 46, wherein composite material comprises a polymer selected from one or more of PI, PC, and PEEK and the secondary material comprises PZT.

48. An aerosol generator core as claimed in claim 47, further comprising electrodes on a surface of the vibration drive.

49. An aerosol generator comprising:
a body having a support supporting an integral membrane and a vibration drive adapted to be connected to an electrical voltage supply
wherein the vibration drive comprises the body, the body being configured to vibrate with application of an electrical voltage across it,
the body comprises a piezoelectric polymer, and
at least one power conductor terminal for delivering electrical power to the vibration drive.

50. The aerosol generator of claim 49, wherein the power conductor terminals comprise at least one spring pin.

51. The aerosol generator of claim 50, wherein the power conductor terminals comprise a spring pin for conducting power to a first vibration drive surface and another spring pin for conducting power to an opposed vibration drive surface via a clip-shaped terminal which extends around the body at a side edge, wherein the clip-shaped terminal comprises a first limb engaged by a spring pin and pressing against an insulator, said first limb being connected to a second limb which provides power to the opposed vibration drive surface.

52. The aerosol generator of claim 51, wherein the power conductor terminals comprise a spring pin for conducting power to a first vibration drive surface and another spring pin for conducting power to an opposed vibration drive surface via a plated through hole.

53. The aerosol generator of claim 50, further comprising at least one electrode on a surface of the vibration drive, wherein the at least one electrode has a characteristic of being applied using sputtering of one or more of copper, nickel, or aluminium.

54. The aerosol generator of claim 53, wherein the at least one electrode has a characteristic of being screen-printed.

55. The aerosol generator of claim 50, wherein a body thickness is between 20 μm and 5000 μm.

56. A method of manufacturing an aerosol generator core comprising:
forming a body by any one or more of hot embossing, micro-moulding, inkjet printing, 3D Printing, or UV Nanoimprint Lithography, wherein
the body comprises a support supporting an integral membrane and a vibration drive adapted to be connected to an electrical voltage supply.

57. The method of claim 56, further comprising hot embossing in which temperature of a polymer sheet is raised above its melting range followed by pressing a heated mould into the polymer to fill surface structures.

58. The method of claim 56, further comprising injection moulding in which polymer granules are melted, and then injected into a mould under pressure where the melted polymer granules solidify.
